(19) United States
(12) Patent Application Publication
(10) Pub. No.: US 2023/0032498 A1
(43) Pub. Date: Feb. 2, 2023

(54) Frame and Method for Constructing Nerve Tract

(71) Applicant: Beijing Institute of Technology, Beijing (CN)

(72) Inventors: Qing Shi, Beijing (CN); Zihou Wei, Beijing (CN); Tao Sun, Beijing (CN); Huaping Wang, Beijing (CN); Zhe Chen, Beijing (CN); Qiang Huang, Beijing (CN)

(21) Appl. No.: 17/527,561

(22) Filed: Nov. 16, 2021

(30) Foreign Application Priority Data
Jul. 27, 2021 (CN) 202110855411.9

(51) Int. Cl. A61M 25/00; C12N 5/079

(52) U.S. Cl. CPC A61M 25/005; C12N 5/0618; A61M 25/0009; A61M 2025/0042; A61M 25/0021

(57) ABSTRACT

A frame for constructing nerve tract is provided, including microcatheters, a support, and a shell. The shell is configured to contain a culture medium inside. The microcatheters are configured to culture nerve cells. The multiple microcatheters are suspended and fixed into the shell by the support, the microcatheters are arranged along a direction from one end to the other end of the shell, catheter walls of the microcatheters are provided with through holes, the nerve cells in the microcatheter cannot flow out through the through holes, and the culture medium enters the microcatheter through the through holes. A method for constructing nerve tract based on the frame described is provided, including: filling the nerve cells wrapped with a collagen hydrogel stock solution into the microcatheters, and after the collagen hydrogel stock solution is completely cross-linked, placing the frame loaded with the nerve cells in a culture device for perfusion culture.

### CROSS REFERENCE TO RELATED APPLICATION

This patent application claims the benefit and priority of Chinese Patent Application No. 202110855411.9, entitled “frame and method for constructing nerve tract” filed with the Chinese Patent Office on Jul. 27, 2021, the disclosure of which is incorporated by reference herein in its entirety.

### TECHNICAL FIELD

The present invention relates to the technical field of tissue engineering, and in particular relates to a frame and a method for constructing nerve tract.

### BACKGROUND ART

Statistically, at present, there are more than 1 billion people in the world to be disturbed by the damage to the nervous system. However, the self-healing capability of the nerve tissues is very weak. Therefore, the methods of constructing nerve tissues in vitro as an implant by tissue engineering, and repairing the damaged nerves by nerve implantation have received widespread attention. Moreover, before the nerve tissues are implanted into the damaged nerves, the nerve tissues constructed in vitro need to undergo drug tests in advance to test the functions such as information conduction characteristics of the constructed nerve tissues. Anatomic analysis shows that the afferent and efferent nerves of the peripheral nervous system are of a tract-like structure that has a complex three-dimensional hierarchy, and formed by assembling multiple ways of nerve fibers together. According to related research reports, highly directional-alignment linearized structural characteristics within the nerve tract play an important role in directional conduction of nerve signals. Therefore, that the test on a multi-way tract-like nerve tissue with a three-dimensional hierarchical structure is obviously more reliable and accurate than the test on a single nerve fiber. However, the existing methods of constructing nerve tissues in vitro are mainly divided into two types, i.e., methods based on cytobiology and methods based on engineering. For the methods based on cell cytobiology, there are the culture of neural spheres through cell self-assembly and the embedment of neural stem cells into other substrates such as matrigel. Through two ideas of constructing organoid models by inducing differentiation, the methods based on cell cytobiology are difficult to precisely control the structure size and phase shape size of tissues, which is not suitable for the construction of multi-way nerve tracts. The methods based on engineering mainly include micro-channels. Micro-channel-based methods are often used to produce a single nerve fiber, which cannot meet the requirements of construction of multi-way nerve tracts, either. Therefore, there is an urgent need for a new type of frame for constructing nerve tract to solve the problems above.

### SUMMARY

The embodiments aim to provide a frame and a method for constructing nerve tract to solve the problems in the prior art, so as to facilitate the production of multi-way nerve tracts.

In order to achieve the foregoing objective, the embodiments provide the following technical solution:

The embodiments provide a frame for constructing nerve tract, including multiple microcatheters, a support, and a shell, where the shell is sleeve-shaped, the shell is configured to contain a culture medium inside, one end of the shell is provided with a culture medium inlet, and another end is provided with a culture medium outlet; and the microcatheters are configured to culture nerve cells inside, the plurality of microcatheters are suspended and fixed in the shell by the support, each of the microcatheters is arranged along a direction from the one end to the other end of the shell, a wall of each of the microcatheters is provided with a plurality of through holes, the nerve cells in the microcatheters do not flow out through the through holes, and the culture medium enters the microcatheters through the through holes.

Preferably, the support is a hollowed-out structure, the support may be fixedly disposed in the shell, and the microcatheters may be embedded and fixed into the support.

Preferably, a perfusion disc is further included, the one end and the other end of the shell may be open, the microcatheters may be parallel with each other, and same ends of the microcatheters may extend out of the support; and a bottom plate of the perfusion disc may be provided with a plurality of perfusion holes, each of the perfusion holes may correspond to a corresponding one of the microcatheters, each of the perfusion holes may be hermetically connected to a corresponding one of the microcatheters, and the perfusion disc may shield the support.

Preferably, an inner surface of the wall of each of the microcatheters may be a hydrophilic surface.

Preferably, the support may be a perfusable crystal structure.

Preferably, the microcatheters, the support, the shell, and the perfusion disc may be of an integrated structure, and the microcatheters, the support, the shell, and the perfusion disc may be integrally manufactured by photolithography.

The present disclosure further provides a method for constructing nerve tract based on the frame described above, including: filling the nerve cells wrapped with the collagen hydrogel stock solution into the microcatheters 3, and after the collagen hydrogel stock solution completely undergoes the cross-linking, placing the frame loaded with the nerve cells in a culture device for perfusion culture.

Preferably, the nervous tract construction frame includes the perfusion disc, the microcatheters are parallel with each other, and same ends of the microcatheters extends out of the support; a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support; enabling the nerve cells to be enveloped by a collagen hydrogel stock solution, and flow into the microcatheters through same ends of the microcatheters connected to the perfusion disc, and removing the perfusion disc.

Preferably, performing the perfusion culture using the frame loaded with the nerve cells in manner of continuous flow in the culture device.

Preferably, putting the frame loaded with the nerve cells into a tip of a pipette, connecting a culture medium inlet of the pipette to a culture medium injector, placing a liquid waste collection dish at a liquid waste outlet of the pipette, and performing the perfusion culture in manner of continuous flow at a flow rate of 200 uL/h to 400 uL/h for one week.

Compared with the prior art, the embodiments have achieved the following technical effects:

The embodiments provide a frame and a method for constructing nerve tract. Each microcatheter suspended and fixed into the shell can cultivate nerve fibers. Therefore, the frame and the method for constructing nerve tract facilitate the production of multi-way nerve tracts.

### BRIEF DESCRIPTION OF THE DRAWINGS

In order to illustrate the technical solutions in the embodiments of the present disclosure or the prior art more clearly, the drawings that need to be used in the descriptions of the embodiments or the prior art will be briefly introduced below. Obviously, the drawings in the following descriptions are merely some embodiments of the present disclosure. For a person of ordinary skill in the art, other drawings may be obtained according to these drawings without creative work.

FIG. 1 is an overall structural diagram of a frame for constructing nerve tract provided in Embodiment 1;

FIG. 2 is a schematic structural diagram of a support and a shell in the frame for constructing nerve tract provided in Embodiment 1;

FIG. 3 is a schematic structural diagram of the support, the shell, and microcatheters in frame provided in Embodiment 1; and

FIG. 4 is a schematic structural diagram of a microcatheter in the frame provided in Embodiment 1.

Reference signs in the drawings: 1 shell, 2 perfusion disc, 21 perfusion hole, 3 microcatheter, 31 through hole, and 4 support.

### DETAILED DESCRIPTION OF THE EMBODIMENTS

The technical solutions in the embodiments of the present disclosure will be clearly and completely described below with reference to the accompanying drawings in the embodiments of the present disclosure. Obviously, the embodiments described are merely a part of the embodiments of the present disclosure, not all the embodiments. Based on the embodiments of the present disclosure, all other embodiments obtained by a person of ordinary skill in the art without creative work shall fall within the protection scope of the present disclosure.

The present disclosure aims to provide a frame and a method for constructing nerve tract to solve the problems in the prior art so as to facilitate the production of multi-way nerve tracts.

In order to make the objectives, features and advantages of the present disclosure clearer and more understandable, the present disclosure will be described in further detail below in conjunction with the drawings and specific implementations.

### Embodiment 1

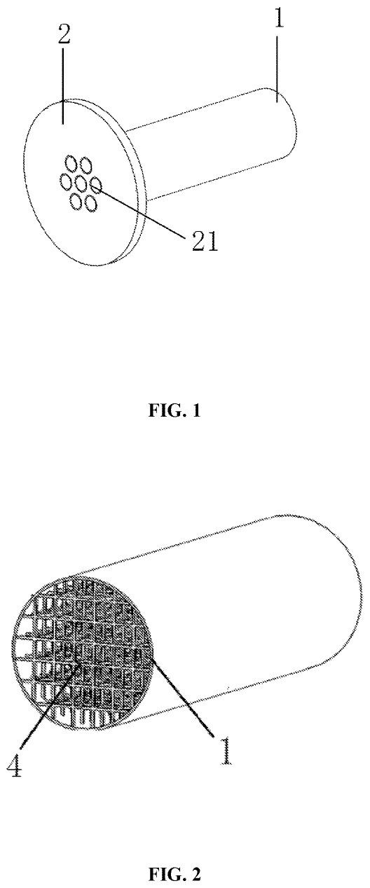

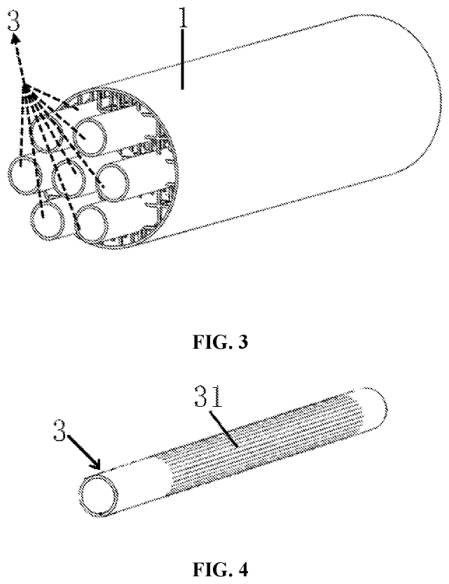

This embodiment provides a nervous tract construction frame, as shown in FIGS. 1-4, including multiple microcatheters 3, a support 4, and a shell 1. The shell 1 is sleeve-shaped. The shell 1 is configured to contain a culture medium inside. One end of the shell 1 is provided with a culture medium inlet, and another end is provided with a culture medium outlet. The microcatheters 3 are configured to cultivate nerve cells. Multiple microcatheters 3 are all suspended and fixed into the shell 1 by the support 4. Each of the microcatheters 3 is arranged along a direction from one end to the other end of the shell 1. Catheter walls of the microcatheters 3 are provided with multiple through holes 31. The nerve cells in the microcatheter 3 cannot flow out through the through holes 31. The culture medium can enter the microcatheters 3 through the through holes 31. Each microcatheter 3 suspended and fixed in the shell 1 can culture nerve fibers. Multiple nerve fibers form a multi-way nerve tract. The multiple nerve fibers are cultured in the shell 1 at the same time, and the nerve tract produced can be directly used in drug test without removing the shell 1 and the support 4. Therefore, the frame and the method for constructing nerve tract provided by the present disclosure facilitate the production of the multi-way nerve tract and can improve the reliability and reality of drug test.

Further, the support 4 is of a hollowed-out structure. The support 4 is fixedly disposed in the shell 1. The microcatheters 3 are embedded and fixed into the support 4. The support 4 of the hollowed-out structure can be perfused with a culture medium. Specifically, the support 4 is a perfusable crystal structure. With the above structure, the stability of the microcatheters 3 can be improved without affecting the perfusion culture of the nerve cells in the microcatheters 3.

Further, a perfusion disc 2 is further included. The one end and the other end of the shell 1 are open. The microcatheters 3 are parallel with each other. Same ends of the microcatheters 3 extends out of the support 4. A bottom plate of the perfusion disc 2 is provided with multiple perfusion holes 21, each of the perfusion hole 21 corresponds to a corresponding one of the microcatheters 3, and each of perfusion hole 21 is hermetically connected to a corresponding one of the microcatheters 3. The perfusion disc 2 shields the support 4. The perfusion disc 2 is provided to prevent the nerve cells and an initial culture material thereof from flowing into the support 4 when the nerve cells and the initial culture material thereof are perfused into the microcatheters 3. With the arrangement above, the accuracy of the perfusion is improved. The initial culture material is a collagen hydrogel stock solution.

Further, an inner surface of the wall of each of the microcatheters 3 is hydrophilic surface. Therefore, when the collagen hydrogel stock solution envelopes with the nerve cells is perfused into the microcatheter 3, the collagen hydrogel stock solution is automatically sucked into the microcatheters 3 under the action of capillary force. Specifically, a plasma is used for hydrophilic treatment. A plasma generator uses PVA Tepla AG GIGA batch 310M, and the plasma is generated with oxygen. A gas flow is set to 100 sccm, a power is set to 300 W, and a processing time is 5 minutes. After the plasma treatment, the frame is placed under an ultraviolet lamp for sterilization for 30 minutes.

Further, the microcatheters 3, the support 4, the shell 1, and the perfusion disc 2 are of an integrated structure. The microcatheters 3, the support 4, the shell 1, and the perfusion disc 2 are integrally manufactured by photolithography with accurate processing dimensions, and specifically are processed by a Photonic Professional GT2 system of Nanoscribe. During processing, a 25× objective lens is used, a photoresist is IP-S photoresist, and a substrate is a glass substrate plated with ITO (Indium tin oxide). The process parameters are set to Slicing 1.0 and Hatching 0.5. A laser power is 80% of the maximum power, a scanning rate is 70,000 um/s, a Z-direction drive mode is selected as Z-drive, and a structure is not divided into blocks. After the processing is completed, the frame for constructing nerve tract is soaked in propylene glycol methyl ether acetate (PGMEA) for development process for 6 hours, and then soaked in isopropanol for 30 minutes before being taken out for use.

Further, in order to provide a sufficient curvature of the guide of the linear growth of nerve fibers, a diameter of each of the microcatheters 3 is designed to be 150 um, preferably seven microcatheters 3. The support 4 of a crystal structure is used as the connection between the seven microcatheters 3. A unit cell of the crystal structure is designed as a cube with a side length of 100 um. A pillar thickness of the unit cell, a wall thickness of each of the microcatheters 3, and a thickness of the shell 1 are all designed to be 10 um. With the design of the crystal structure, the support 4 is perfusable. The array arrangement of micropores on a side wall of the microcatheter 3 plays a role in promoting material exchange between nerve tissues in the microcatheters 3 and the culture medium, so as to bring effects of better perfusion culture. A diameter of each of the through holes 31 is 5 um. Since an average diameter of the cell body of each of the nerve cells is 10 um or more, the through holes 31 in the side walls of the microcatheters 3 allows macromolecular nutrients such as glucose and protein to pass through, thereby preventing the nerve cells in the microcatheters 3 from migrating to the outside of the microcatheters 3. Since there are a large number of holes in the side walls of the microcatheters 3, which causes stress concentration. So, every three adjacent holes in the each microcatheter 3 are in a triangular arrangement. The structural design above can relieve the stress concentration to a certain extent.

### Embodiment 2

The present embodiment provides a method for constructing nerve tract based on the frame described in Embodiment 1, including: filling the nerve cells wrapped with the collagen hydrogel stock solution into the microcatheters 3, and after the collagen hydrogel stock solution completely undergoes the cross-linking, placing the frame loaded with the nerve cells in a culture device for perfusion culture. The cross-linking refers to a process the collagen hydrogel stock solution changes from the liquid to gel.

Further, the frame includes the perfusion disc 2, all the microcatheters 3 are parallel with each other, and same ends of the microcatheters 3 extends out of the support. The bottom plate of the perfusion disc 2 is provided with multiple perfusion holes 21. Each of the perfusion hole 21 corresponds to a corresponding one of the microcatheters 3, and each of the perfusion hole 21 is hermetically connected to a corresponding one of the microcatheters 3. The perfusion disc 2 shields the support, the nerve cells is enveloped by a collagen hydrogel stock solution, and flow into the microcatheters through one end of each of the microcatheters connected to the perfusion disc, and then the perfusion disc is removed.

Further, the perfusion culture is performed using the frame loaded with the nerve cells in manner of continuous flow in the culture device.

Further, the frame loaded with the nerve cells is put into a tip of a pipette, a culture medium inlet of the pipette is connected to a culture medium injector, a liquid waste collection dish is placed at a liquid waste outlet of the pipette. And the frame loaded with the nerve cells is cultured under the continuous flow perfusion at a flow rate of 200 uL/h to 400 uL/h for one week. The continuous perfusion culture is to simulate a real culture environment of nerve fibers in human body to improve the biomimeticity. Furthermore, metabolic wastes of the nerve cells can flow out of the microcatheters through the through holes to reduce the concentration of metabolic wastes in the growth environment of the nerve cells.

The method for constructing nerve tract is specifically as follows.

First, the collagen hydrogel stock solution is prepared, where a specific method for preparing the collagen hydrogel stock solution is: 10 mg of rat tail collagen is weighed and dissolved in a 0.2% glacial acetic acid solution. Then, 100 uL of collagen solution is taken and 36 uL of 0.1 M NaOH solution is added thereto for neutralizing. 15 uL of 10×PBS (Phosphate Buffered Saline) is added to adjust the ion salt concentration. Finally, 49 uL of nerve cell culture medium is added, where the composition of the nerve cell culture medium is a RPMI (Roswell Park Memorial Institute) 1640 with 10% horse serum, 5% fetal Bovine serum, and 1% penicillin and streptomycin added. After the preparation, the collagen hydrogel stock solution envelopes with nerve cells, where the nerve cells are PC12 (Pheochromocytoma-derived cell line). A specific method is as follows: the PC12 cells overgrowing in a culture dish are passaged, and the centrifuged cell suspension is diluted 10 times, and then the cell suspension is centrifuged again. The collagen hydrogel stock solution filtered by a 0.22 um microporous membrane is added to cell sediments obtained by centrifugation. Then, pipetting and suspending are performed to obtain the collagen hydrogel stock solution enveloping with the nerve cells. After the collagen hydrogel stock solution is perfused into the microcatheters 3, the microcatheters are allowed to stand at room temperature for 15 minutes, so as to wait for adding the culture medium after the collagen is pre-crosslinked. The microcatheters are put into a 37° C. incubator for standing for 1 hour until the collagen is completely cross-linked. Then, the microcatheters are taken out and the perfusion disc 2 is peeled off with tweezers. Next, the porous bionic frame loaded with cells is carefully inserted into the tip of a pipette with a 10 uL of flow rate. One end of each of the micropipettes 3 can be set in a closed state. The nutrient solution in the tip of the pipette flows along the direction from the closed ends of the microcatheters 3 to open ends thereof. The porous bionic frame is gently pushed by using a capillary glass tube until the frame is stably fixed to the tip of the pipette. Then, this frame is placed in the culture device on a microscope. An inlet of the tip of the pipette is connected to an injector, and a culture dish is placed at an outlet of the tip of the pipette to collect waste liquid. The perfusion culture is carried out for one week at a flow rate of 300 uL/h to obtain multi-way nerve tracts with high cell viability, high cell density, and linear growth.

Specific examples are used in the present disclosure to illustrate the principles and implementations of the present disclosure. The descriptions of the foregoing embodiments are only for assisting understanding the method and core ideas of the present disclosure. In the meantime, for a person of ordinary skill in the art, according to the spirit of the present disclosure, there will be some modifications to the specific implementations and scope of application. In summary, the content of this specification should not be construed as a limitation to the present disclosure.

What is claimed is:

1. A frame for constructing nerve tract, the frame comprising a plurality of microcatheters, a support, and a shell, wherein the shell is sleeve-shaped, the shell is configured to contain a culture medium inside, one end of the shell is provided with a culture medium inlet, and another end of the shell is provided with a culture medium outlet; and the microcatheters are configured to culture nerve cells inside, the plurality of microcatheters are suspended and fixed in the shell by the support, each of the microcatheters is arranged along a direction from the one end to the other end of the shell, a wall of each of the microcatheters is provided with a plurality of through holes, the nerve cells in the microcatheters do not flow out through the through holes, and the culture medium enters the microcatheters through the through holes.

2. The frame according to claim 1, wherein the support is of a hollowed-out structure, the support is fixedly disposed in the shell, and the microcatheters are embedded and fixed into the support.

3. The frame according to claim 2, further comprising a perfusion disc, wherein the one end and the other end of the shell are open, the microcatheters are parallel with each other, and same ends of the microcatheters extend out of the support; and a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support.

4. The frame according to claim 1, wherein an inner surface of the wall of each of the microcatheters is a hydrophilic surface.

5. The frame according to claim 2, wherein the support is a perfusable crystal structure.

6. The frame according to claim 3, wherein the microcatheters, the support, the shell, and the perfusion disc are of an integrated structure, and the microcatheters, the support, the shell, and the perfusion disc are integrally manufactured by photolithography.

7. A method for constructing nerve tract based on the frame, the frame comprising a plurality of microcatheters, a support, and a shell, wherein the shell is sleeve-shaped, the shell is configured to contain a culture medium inside, one end of the shell is provided with a culture medium inlet, and another end of the shell is provided with a culture medium outlet; and the microcatheters are configured to culture nerve cells inside, the plurality of microcatheters are suspended and fixed in the shell by the support, each of the microcatheters is arranged along a direction from the one end to the other end of the shell, a wall of each of the microcatheters is provided with a plurality of through holes, the nerve cells in the microcatheters do not flow out through the through holes, and the culture medium enters the microcatheters through the through holes;
the method, comprising: filling the nerve cells into the microcatheters, and placing the frame loaded with the nerve cells in a culture device for perfusion culture.

8. The method according to claim 7, wherein the support is of a hollowed-out structure, the support is fixedly disposed in the shell, and the microcatheters are embedded and fixed into the support.

9. The method according to claim 8, wherein the frame further comprising a perfusion disc, wherein the one end and the other end of the shell are open, the microcatheters are parallel with each other, and same ends of the microcatheters extend out of the support; and a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support.

10. The frame according to claim 7, wherein an inner surface of the wall of each of the microcatheters is a hydrophilic surface.

11. The frame according to claim 8, wherein the support is a perfusable crystal structure.

12. The frame according to claim 9, wherein the microcatheters, the support, the shell, and the perfusion disc are of an integrated structure, and the microcatheters, the support, the shell, and the perfusion disc are integrally manufactured by photolithography.

13. The method according to claim 7, wherein the frame comprises a perfusion disc, the microcatheters are parallel with each other, and same ends of the microcatheters extends out of the support; a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support;
enabling the nerve cells to be enveloped by a collagen hydrogel stock solution, and flow into the microcatheters through one end of each of the microcatheters connected to the perfusion disc, and
removing the perfusion disc.

14. The method according to claim 8, wherein the frame comprises a perfusion disc, the microcatheters are parallel with each other, and same ends of the microcatheters extends out of the support; a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support;
enabling the nerve cells to be enveloped by a collagen hydrogel stock solution, and flow into the microcatheters through one end of each of the microcatheters connected to the perfusion disc, and
removing the perfusion disc.

15. The method according to claim 9, wherein enabling the nerve cells to be enveloped by a collagen hydrogel stock solution, and flow into the microcatheters through same ends of the microcatheters connected to the perfusion disc, and
removing the perfusion disc.

16. The method according to claim 10, wherein the frame comprises a perfusion disc, the microcatheters are parallel with each other, and same ends of the microcatheters extends out of the support; a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support;
enabling the nerve cells to be enveloped by a collagen hydrogel stock solution, and flow into the microcatheters through one end of each of the microcatheters connected to the perfusion disc, and
removing the perfusion disc.

17. The method according to claim 11, wherein the frame comprises a perfusion disc, the microcatheters are parallel with each other, and same ends of the microcatheters extends out of the support; a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support;
enabling the nerve cells to be enveloped by a collagen hydrogel stock solution, and flow into the microcatheters through one end of each of the microcatheters connected to the perfusion disc, and
removing the perfusion disc.

18. The method according to claim 12, wherein the frame comprises a perfusion disc, the microcatheters are parallel with each other, and same ends of the microcatheters extends out of the support; a bottom plate of the perfusion disc is provided with a plurality of perfusion holes, each of the perfusion holes corresponds to a corresponding one of the microcatheters, each of the perfusion holes is hermetically connected to a corresponding one of the microcatheters, and the perfusion disc shields the support;
enabling the nerve cells to be enveloped by a collagen hydrogel stock solution, and flow into the microcatheters through one end of each of the microcatheters connected to the perfusion disc, and
removing the perfusion disc.

19. The method according to claim 7, wherein performing the perfusion culture using the frame loaded with the nerve cells in manner of continuous flow in the culture device.

20. The method according to claim 19, wherein putting the frame loaded with the nerve cells into a tip of a pipette, connecting a culture medium inlet of the pipette to a culture medium injector, placing a liquid waste collection dish at a liquid waste outlet of the pipette, and performing the perfusion culture in manner of continuous flow at a flow rate of 200 uL/h to 400 uL/h for one week.
